(19) United States
(12) Patent Application Publication
(10) Pub. No.: US 2003/0215465 A1
(43) Pub. Date: Nov. 20, 2003

(54) Use of conjugated acid derivatives

(75) Inventors: Frederick William Cain, Wormerveer (NL); Ulrike Schmid, Wormerveer (NL); Marianne O'Shea, Channahon, IL (US); Ingrid Celestina Maria Mohede, Wormerveer (NL)

Correspondence Address: MORGAN LEWIS & BOCKIUS LLP, 1111 PENNSYLVANIA AVENUE NW, WASHINGTON, DC, 20004, US

(73) Assignee: LC USA LLC

(21) Appl. No.: 10/417,349

(22) Filed: Apr. 17, 2003

(30) Foreign Application Priority Data
Apr. 25, 2002 (EP) 02076639.0

(51) Int. Cl. A61K 39/21; A61K 9/48; A61K 31/202

(52) U.S. Cl. 424/208.1; 514/560; 424/452

(57) ABSTRACT

The invention concerns the use of conjugated linoleic acid (=CLA) or derivatives thereof, such as partial glycerides or triglycerides, alkyl esters or salts for the production of a food, a food supplement or a pharmaceutical preparation with the property to prevent or to cure influenza, to boost the effects of an influenza vaccination and/or to alleviate the effects of an influenza vaccination in humans and/or animals.

This invention concerns in its broadest sense the use of Conjugated Linoleic Acid (=CLA) derivatives as an additive or as a component for foods, food supplements or pharmaceutical preparations which provide these foods, food supplements or pharmaceutical preparations with a specific health effect. From U.S. Pat. No. 6,020,376 it is known that CLA can be used to maintain or to elevate CD-4 and CD-8 cell levels in animals to boost or benefit their immune system. However this document does not reveal a direct link with what type of disease can benefit from this use of CLA. As a certain ratio of CD-4 and CD-8 cells plays a role in the occurrence and/or treatment of many different types of diseases it remains unclear whether all these types of diseases can be prevented/cured by the use of CLA. This document further reveals that CLA can be used to alleviate the weight loss and other adverse effects from the production and exogeneous administration of TNFa in animals or humans or from the infection of animals or humans by viruses. According to column 9 lines 1 to 15 CLA can be used against infections by a number of different families of viruses. Although it is known that one group member of one of the viruses mentioned (=Orthomyxovirus family) comprise also the influenza virus genus, it cannot be derived from this document that CLA will have beneficial activity against influenza infections because other family members or serogroups of the same family are known to have other activities. Orthomyxovirus consists of two family members i.e. Thogoto like viruses and influenze viruses. Thogoto like viruses include Thogoto viruses, Dhori viruses and Batken viruses. These three genera generally are considered as three serogroups of the Thogoto like viruses. They clearly differ from influenza viruses on the following points:

Thogoto like viruses are transmitted via ticks and some vertebrates, the influenza viruses are transmitted in droplets as people sneeze, cough or talk, facilitated by close contact

the mechanism of actions differ for the Thogoto like viruses from that of the influenza viruses. In contrast to the influenza viruses the Thogoto viruses are not applying the classical cap snatching mechanism

the nature of the illness caused by the different viruses is different. Thogoto viruses leading to a more severe illness than influenza viruses such as optic neuritis and fatal meningitis.

Because of these basic differences in mechanism a man skilled in the art never would have expected that CLA could have a positive effect on all the family members of the whole Orthomyxovirus family.

U.S. Pat. No. 5,827,885 being the mother patent of above U.S. Pat. No. 376 has a similar teaching although the claims now are limited to the anti viral effects of CLA. Again influenza is not disclosed in this document. Here the same argument as above will account.

According to XP-002217292 (publication from Kelley c.s in Lipids 35, no 10, 2000, pages 1065-1071) CLA has an immunizing activity on animals which however was not found to exist for healthy young women. Therefore this publication is teaching away from using CLA or its derivatives for use as anti influenza agent for humans.

U.S. Pat. No. 5,674,901 discloses a similar teaching as above two US patents although the claims are limited to maintenance of CD-4 and CD-8 cell levels. So again no teaching is given that CLA would be beneficial against influenza.

As influenza is a known disease for already a very long time and no good means are known to prevent influenza, apart from an inactive influenza vaccination (for which humans sometimes are not always sensitive so that they develop as a reaction hereon insufficient amounts of anti-bodies to prevent them from suffering from influenza) there exists a great need to find compounds that would help to a higher extent than known so far to prevent/cure/treat humans suffering from influenza . A particular benefit is obtained by the fact that we found that the effects of an inactive influenza vaccination can be boosted by our compounds i.e., humans after influenza vaccination who also use in combination therewith our components will develop anti-bodies against influenza to a greater extent and in a shorter time than humans which are injected only.

Therefore our invention concerns in the first instance the use of conjugated linoleic acid (=CLA) or derivatives thereof, such as partial glycerides or triglycerides, alkyl esters or salts, wherein the CLA or derivative thereof is used for the production of a food, a food supplement or a pharmaceutical preparation with the property to prevent or to cure influenza, to boost the effects of an influenza vaccination and/or to alleviate the effects of an influenza vaccination in humans. The food that is made by our invention can contain a wide range of amounts of CLA. In practice effective amounts will be present in the food. Effective being defined as that amount that gives a noticeable positive effect. We however prefer to make foods that comprise from 0.1 to 20 grams of CLA derivative per daily portion. So if the daily portion is just one item of food this item will contain the total amount of CLA, if however the daily portion is used in different portion during the whole day each portion will contain the reciprocal amount of CLA. It will be obvious from above that the CLA derivative is used for the production of a food, a food supplement or a pharmaceutical preparation with the ability to prevent or to cure influenza, caused by a virus of the influenza virus genus including the different serogroups.

Although about every type of food product can be made according to the invention we have a preference for the production of a food selected from the group consisting of margarine; fat continuous or water continuous or bicontinuous spreads, fat reduced spreads, confectionery products such as chocolate or chocolate coatings or chocolate fillings or bakery fillings; ice creams; ice cream coatings; ice cream inclusions; dressings, mayonnaises, cheeses, cream alternatives, dry soups, drinks, cereal bars, sauces, snack bars, dairy products, clinical nutrition and infant formula and having the ability to prevent or to cure influenza caused by a virus, in particular by a virus from the influenza virus genus including the different serogroups.

In the alternative we also can formulate our new invention as a method for administering a CIA derivative to a human suffering from influenza or to a human having the intention to prevent influenza by administering to this human an effective daily dosage of a CLA derivative (free acid/mono-, di- or tri-glycerides/alkyl esters, for example wherein the alkyl group contains from 1 to 12, more preferably 1 to 6, carbon atoms/salts, for example sodium salts). We have a preference for a method wherein the CIA derivative is administered in the form of a food or a food supplement comprising an effective amount of CLA per portion, in particular 0.1 to 20 gram per daily portion. In particular we prefer a method wherein the CLA derivative is administered to a human suffering from influenza or trying to prevent influenza caused by a virus from the influenza virus genus including the different serogroups by administering an effective daily dosage of CLA-derivative per portion, in particular 0.1 to 20 gram per daily portion. The method may comprise a method of treating and/or preventing influenza.

Although we can use the different known isomers of CLA we prefer to use the isomers cis9trans11 and trans10cis12 and in particular mixtures of these isomers wherein the isomers are present in ratios of 80:20 to 20:80 and most preferably isomer mixtures wherein the trans10cis12 isomer is present for more than 60 wt %

In the instance that a food supplement is administered we prefer to supply the food supplement as a capsule in the form of a soft gel or a hard capsule wherein the encapsulating material comprises a material selected from the group consisting of gelatine; starch; modified starch; modified starch flour, sugars, in particular sucrose; lactose and fructose.

In the instance that a pharmaceutical preparation is administered we prefer to supply the pharmaceutical preparation in the form of a tablet, capsule, solution or emulsion depending upon the form of CLA employed and the route of administration.

According to a last embodiment of our invention we found that the CLA derivatives are most active when administered to elderly women (i.e., women with an age of at least 55 years) or to men.

### EXAMPLES

### Example 1

The resistance inducing effect of CLA against viral airway infections (prevention) was investigated/evaluated in rats. Rats were fed with CIA for 5 weeks before exposure (intranasal) to the influenza virus. The route of exposure is similar to the route humans are exposed to the influenza virus. The samples taken at several time points reflect the development of the infection and the protective effect by CLA.

Dosage=1% CLA (FFA)

The following parameters were measured:

growth

food intake

viral particles in lungs

Influenza specific antibodies

Th1 (IFN-gamma) and Th2 (IL10) cytokines

Macrophage cytokine (IL1)

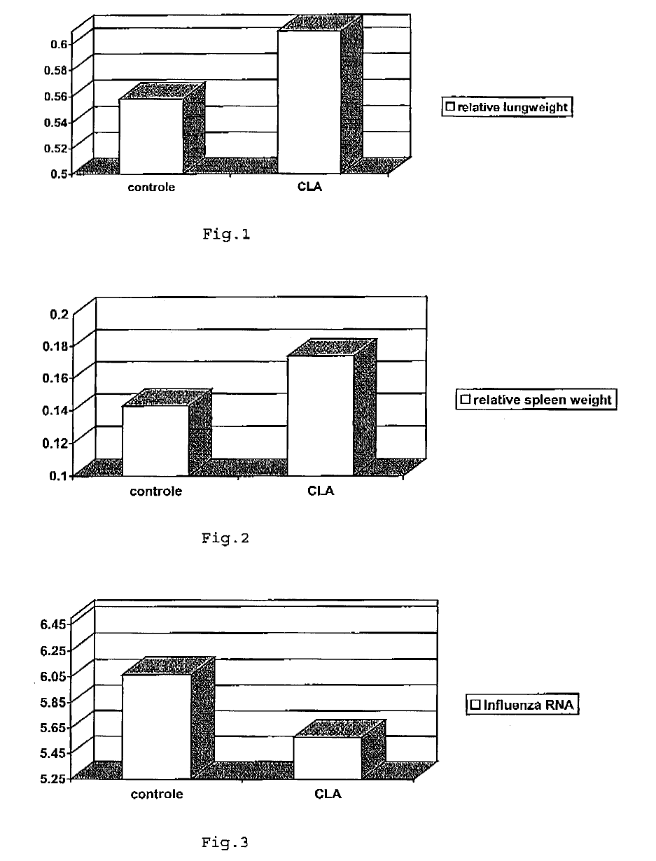

The results are shown in FIGS. 1, 2 and 3.

FIG. 1 shows that for rats fed with CLA lungweight is increased due to infiltration of immune cells from the immune organs (for example the spleen) to the target (lung). The immune cells can attack the virus (directly via macrophages or indirectly as initiated via cytokines).

FIG. 2 shows the effect on the spleen. The spleen is a central immune organ. The spleen will increase in weight due to the increased amount of immune cells which are activated. Those immune cells will migrate to the target (lung) due to the infection.

FIG. 3 shows the effect of CLA on influenza RNA. The influenza virus (expressed in viral particles (single-stranded RNA)) is cleared by the immune cells. This clearance occurs directly (macrophages) or indirectly (initiated by cytokines)

### Example 2

Evaluation of the adjuvant effect of dietary supplementation with conjugated linoleic acid (CLA) on the immune response to influenza vaccine in healthy elderly subjects.

CLA was provided in capsules containing 1.25 g (1 g of active isomers) of a 50:50 mixture of the two isomers c9, t11 and t10, c12 CLA in either free fatty acid form (A-80) or triglyceride form (G-80). Placebo was provided in identical capsules containing high-oleic sunflower oil (HOSF). Two capsules (2 g of active isomer) were taken once daily with food for 49 days. Subjects were given a two week supply of individually packaged supplement or identical placebo on days 1, 14, 28, and 42.

The study was conducted on healthy, elderly (≧65 years old) adults.

The trial was conducted as a randomized, double-blind study of A-80, C-80, or control. All subjects received a single dose of inactivated influenza vaccine in open-label fashion.

The volunteers received either A80/G80 or HOSF for 28 days before vaccination. A single dose of inactivated influenza vaccine was administered intramuscularly on day 28 (±3). The ability of CLA to adjuvant the immune response to influenza vaccine was assessed by comparison of the HAI antibody response to each of the three components of the vaccine between individuals who did or did not receive nutritional supplementation.

Subjects were classified as responders and non-responders for antibody production. For antibody production, subjects were considered responders when titers were above a certain titer as these levels are considered to be protective against Influenza infection.

Comparison of the Effect of CLA in Free Fatty Acid (A-80) or Triglyceride (G-80) Formulations

The overall response rates are shown in the table below

| | number of | | | |
|---|---|---|---|---|
| treatment | people | H1N1 | H3N2 | B |
| A80 | 22 | 50.0% | 45.5% | 90.0% |
| HOSF | 27 | 33.3% | 37.0% | 63.3% |
| G80 | 23 | 21.7% | 21.7% | 78.3% |

A vaccine contains 3 components (2 derived from Influenza A (expressed as H1N1 and H3N2), 1 from Influenza n)

The vaccination composition was:

H1N1: A/New Caledonia/20/99

H3N2: A/Moscow/10/99

B: B/Hong Kong/330/01

The conclusions that can be drawn from these results are:

A80 increased the antibody response rates against all components of the influenza vaccination compared with control.

G80 increased the antibody response rate against influenza B.

1. Method for preventing or curing influenza, or boosting the effects of an influenza vaccination and/or alleviating the effects of an influenza vaccination in humans comprising administering a CLA derivative to a human suffering from influenza or to a human having the intention to prevent influenza by administering to this human an effective daily dosage of a CLA derivative (free acid/glycerides/alkyl esters/salts).

2. Method according to claim 1 wherein the CLA derivative is administered in the form of a food or a food supplement comprising an effective amount of CLA per portion, in particular 0.1 to 20 gram per daily portion.

3. Method according to claim 1 wherein the CLA derivative is administered to a human suffering from influenza or trying to prevent influenza caused by a virus from the influenza virus genus including the different serogroups.

4. Method according to claim 1 wherein the effective dose that is administered is 0.1 to 20 g per day.

5. Method according to claim 2 wherein the food supplement is a capsule in the form of a soft gel or a hard capsule wherein the encapsulating material is selected from the group consisting of gelatine; starch; modified starch; modified starch flour, sugars, in particular sucrose; lactose and fructose.

6. Method according to claim 1 wherein the pharmaceutical preparation is in the form of tablet, capsule, solution or emulsion depending upon the form of CLA employed and the route of administration.

7. Method according to claim 1 wherein the CLA derivative is administered to elderly women (i.e., women having an age of 55 years or more) or to men.
